(19) United States
(12) Patent Application Publication
(10) Pub. No.: US 2025/0090373 A1
(43) Pub. Date: Mar. 20, 2025

(54) OPHTHALMIC INJECTION DEVICE

(71) Applicant: David C. Metrikin, El Paso, TX (US)

(72) Inventor: David C. Metrikin, El Paso, TX (US)

(21) Appl. No.: 18/886,341

(22) Filed: Sep. 16, 2024

Related U.S. Application Data
(60) Provisional application No. 63/538,733, filed on Sep. 15, 2023.

(51) Int. Cl. A61F 9/00

(52) U.S. Cl. CPC A61F 9/0017

(57) ABSTRACT

An ophthalmic injection device is disclosed. The injection device is configured to inject a quantity of local anesthetic into or immediately beneath the bulbar conjunctive to create a patch of anesthetized eye wall prior to an intravitreal or subretinal injection. The device is designed to allow complication-free injection of local anesthesia into the eye wall by non-physician healthcare personnel with minimal training.

### REFERENCE TO RELATED APPLICATIONS

This application claims priority of U.S. Provisional Application No. 63/538,733 entitled “Opthalmic Injection Device” filed on 15 Sep. 2023, the disclosures of which are incorporated herein by reference in their entirety.

### BACKGROUND

### Technical Field

The disclosures herein relate to the field of ophthalmic procedures. Specifically, various embodiments of the disclosed invention relate to a subconjunctival injection device and a method of use.

### State of the Art

Over the past decade, the utilization of intravitreal injections (IVI's) has experienced a significant surge, primarily due to the emergence of novel therapeutic agents that have altered the ophthalmologist's ability to treat common retinal conditions. In 2022, over 7 million IVIs were performed in the United States and projections indicate this number will rise.

This surge in IVI utilization has imposed a burden on physician practices compelling them to efficiently accommodate the escalating caseload. Before performing an IVI it is desirable to fully anesthetize the conjunctiva by inducing passive diffusion of anesthetic agents through the tenons, sclera, and choroid. It is estimated that approximately 25% of IVI's necessitate a subconjunctival injection of a local anesthetic medication such as lidocaine before the IVI procedure. In the United States, this injection is largely performed by physicians, largely due to concerns about potential complications arising from inadvertent needle injuries during involvement of a non-physician healthcare provider, such as an advanced practice nurse or other clinical staff. Although a physician must perform the IVI, administering local anesthesia should not necessarily require physician participation. With current conjunctival injection techniques, however, there remains a risk of penetrating the ocular globe with the injection needle. This can result in injury to the globe, intraocular bleeding, and other potentially serious complications. As a result, the current approach necessitates the physician's participation in a two-step process-injecting local anesthesia, then performing the IVI-including a waiting period between these two steps.

In view of the pressing need to optimize efficiency of subconjunctival anesthesia administration and streamlining the subsequent IVI procedure, an ophthalmic injection device designed for easy and safe local administration of a small dose of anesthetic medication into or beneath the bulbar conjunctiva as a precursor to the delivery of the IVI medication is needed. Such a device would enable non-physicians to administer subconjunctival anesthesia, effectively eliminating the requirement for physician participation in establishing local anesthesia prior to the IVI and markedly increasing the number of IVI injection procedures which can be safely performed by a physician over a scheduled time period.

### BRIEF SUMMARY

Disclosed herein are embodiments of ophthalmic injection device having a medicament reservoir, a curved length-tipped needle, and a surface for contacting the conjunctiva.

According to at least one exemplary embodiment, an ophthalmic injection device comprises a body, a reservoir, and a needle. The body includes a flange at one end and a shoulder at the other end. The needle passes through the body and fluidly couples the reservoir, which is mounted on the shoulder of the support, to a curved length configured to project, extend or be deployed external to a contact surface of the flange.

In an alternative embodiment, the needle is embedded in the support. The reservoir and a needle are capable of rotating with the support, ensuring the capture of the conjunctiva. In some embodiments, the ophthalmic injection device future includes an orientation marking indicating the direction of rotation on a dorsal surface of the flange. In some embodiments, the ophthalmic device includes a marking ink disposed on the contact surface of the flange such that the injection site is marked for identification of the IVI site. In some embodiments, the curved length protrudes no greater than about 100 micrometers from the contact surface of the flange, wherein the needle curved length may penetrate the conjunctiva to a sufficient depth for injection of the local anesthetic but penetration into deeper layers of the globe are prevented.

Benefits of the ophthalmic injection device over the use of syringes and other injection devices from the prior art include accurate subconjunctival injection of a local anesthetic at the appropriate depth by a non-surgeon healthcare professional. The curve-shaped needle tip allows for oblique injection of the ocular wall at a set depth. The set depth reduces the risk of ocular injury or inaccurate depth-placement of a local anesthetic. The set oblique angle reduces leakage of the injected medicament from the injection site after removal of the curved length-tipped injection needle. The use of rotation “captures” the conjunctiva and reduces the likelihood of movement of the needle tip during the injection. Further, the combination of the bulb-shaped medication reservoir and device rotation to capture the conjunctive and accurate set the needle tip depth allows a non-surgeon provider to easily perform the injection using only one hand.

In some embodiments, the ophthalmic injection device incorporates marking ink disposed on the contact surface configured to leave a spot of nontoxic marking stain or ink on the outer surface of the conjunctiva to mark the site of local anesthetic injection. As used herein “marking ink” means any non-toxic medical grade stain, ink, or similar marking material that is visible when applied to the bulbar conjunctiva. In some embodiments, the marking ink is sterile. Deposition of the marking ink on the conjunctival surface during use of the ophthalmic injection device permits the physician performing the subsequent IVI to see exactly where the globe has been anesthetized by auxiliary staff for penetration by the IVI needle. Rapid, unambiguous identification of such markings post injection of local anesthesia facilities precise localization of the penetration site for IVI.

Disclosed is an ophthalmic injection device comprising a body with an elongate shape having a flange disposed at a first end and a shoulder disposed a second end, the flange having a dorsal surface and a contact surface opposite the dorsal surface; a pliable reservoir coupled to the shoulder; and a needle coaxial with the body, the needle fluidly coupled to the reservoir and extending through the flange, wherein a curved length of the needle projects external to the contact surface.

In some embodiment, the needle is radially fixed to the body such that rotation of the body causes a corresponding rotation of the needle. In some embodiments, the needle is not radially fixed to the body such that rotation of the reservoir causes rotation of the needle independent of the body and the flange contact surface. In some embodiments, the flange dorsal surface bears an indicia indicating a direction of rotation to cause the needle to penetrate a tissue abutting the contact surface.

In some embodiments, the device further comprises an orientation marking ink on the contact surface. In some embodiments, the orientation marking ink is disposed at two locations on the contact surface at a location proximate to the needle curved length. In some embodiments, the orientation marking ink is disposed at three locations on the contact surface proximate to the needle curved length.

In some embodiments, the curved length projects no greater than about 100 micrometers external to the flange contact surface. In some embodiments, body, the shoulder, and the flange are formed as a unitary structure.

In some embodiments, the reservoir is pre-loaded with a medicament. In some embodiments, the medicament is a local anesthetic. In some embodiments, a volume of the medicament is greater than or equal to about 0.1 milliliters and less than or equal to about 0.2 milliliters.

In some embodiments, the contact surface is curvilinear. In some embodiments, the flange has a diameter of about 75 millimeters. In some embodiments, the curved length of the needle is a 30 gauge needle. In some embodiments, the curved length of the needle is a 31 gauge needle.

Disclosed is an ophthalmic injection device comprising a pliable reservoir pre-loaded with a local anesthetic medicament; a curved length of a needle fluidly coupled to the reservoir and fixedly coupled to the reservoir; and a body rotatably coupled to the reservoir and having a curved contact surface; wherein fixing the contact surface and rotating the reservoir causes the curved length to enter a bulbar conjunctiva of an eye for injection of the medicament.

Disclosed is a method of using an ophthalmic injection device comprising applying a marking ink to a contact surface of an ophthalmic injection device containing a medicament; placing the contact surface of the body of the ophthalmic injector device against an external surface of the eye; rotating the ophthalmic injector device; injecting the medicament; and removing the ocular injection device.

In summary, the ophthalmic injection device features a needle having a curved length for precise injection localization. The rotational approach for conjunctival capture, ensures precise positioning of the local anesthetic injection site. Additionally, the injection site is marked with ink, in some embodiments, to facilitate post-anesthetic injection site identification by physicians or non-physicians for subsequent IVI or other treatment. Disclosed example methods of use may be performed by non-surgeon staff after minimal training and can be rapidly executed, thereby increasing patient flow and improving the ocular surgeon's practice efficiency.

### BRIEF DESCRIPTION OF THE DRAWINGS

FIG. 1 is a side cutaway view of an ophthalmic injection device;

FIG. 2 is a top view of an ophthalmic injection device;

FIG. 3 is a bottom view of an ophthalmic injection device; and

FIG. 4 is a diagram of a method of use of an ophthalmic injection device.

### DETAILED DESCRIPTION

Aspects of the invention disclosed in the following description and related drawings are directed to specific embodiments disclosing features and aspects of the invention. Alternate embodiments may be devised without departing from the spirit or the scope of the invention. Additionally, well-known elements of certain embodiments may not necessarily be described in detail or will be omitted so as not to obscure the relevant details of the invention.

To facilitate an understanding of the description discussion of several terms used herein follows.

As used herein, the word “exemplary” means “serving as an example, instance or illustration.” The embodiments described herein are not limiting, but rather are offered as examples. The described embodiments are not necessarily to be construed as preferred or advantageous over other embodiments. Moreover, the terms “embodiments of the invention,” “embodiments,” or “invention” do not require that all embodiments of the invention include the discussed feature, advantage, or mode of operation.

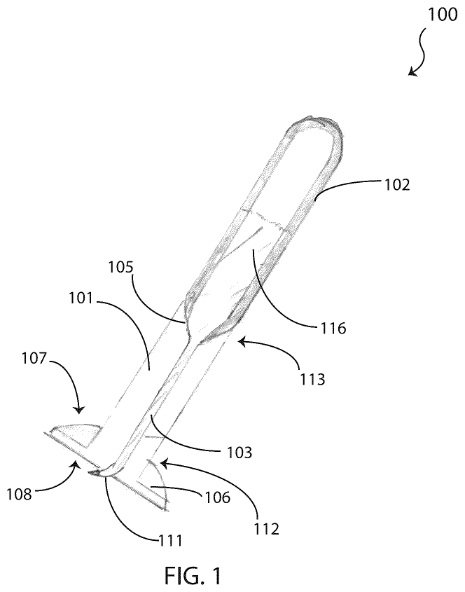

FIGS. 1-3 show exemplary embodiments of an ophthalmic injection device. An ophthalmic injection device having a curved length-tipped needle at a distal end contacting the eyeball enables the user to accurately deliver a local anesthetic injection into the conjunctiva. After contacting the distal end of the device to bulbar conjunctival surface of the ocular globe, rotating the device causes the short, curved length of the needle to penetrate the conjunctiva at an angle which limits the depth to which the needle penetrates the eye wall. In some embodiments a marking ink is disposed on the device-eyeball contact surface to facilitate precise identification of the injection site by the ocular surgeon.

FIG. 1 is a side cutaway view of an example embodiment of an ophthalmic injection device. FIG. 1 shows an ophthalmic injection device 100. Device 100 comprises a body 101, a medicament reservoir 102, and a needle 103. In some embodiments, body 101 includes a shoulder 105 disposed at a first (proximal) end 113 and a flange 106 disposed at a second (distal) end 112. Body 101 is generally elongated, as shown in FIG. 1 and has a circular cross section, in some embodiments. In some embodiments, body 101 is about 150 millimeters (mm) long. In some embodiments, body 101 is longer than 150 mm. In some embodiments, body 101 is shorter than 150 mm. In some embodiments, shoulder 105 and flange 106 form body 101 as a unitary structure. In some embodiments, flange 106 is detachable from body 101. Body 101 may be formed from any suitable inert, medical-grade plastic polymer suitable for a disposable medical injection device known in the art. The medical-grade plastic polymer is substantially rigid, in some embodiments.

Shoulder 105 supports a reservoir 102. Reservoir 102 presents a complementary shape to shoulder 105, upon which it rests. Substantially rigid shoulder 105 supports reservoir 102. Reservoir may be shaped as a generally spherical, generally cylindrical, or other shape, in some embodiments. FIG. 1 also shows needle 103, which passes through body 101, centrally along or generally parallel to a longitudinal axis of body 101, in some embodiments. Needle 103 is hollow and fluidly couples an interior chamber of reservoir 102 to a curved length 111.

In some embodiments, Reservoir 102 is formed from a plastic polymer, silicone rubber, or other suitably pliable, medical-grade material which can be compressed between the user's fingers, collapsing reservoir 102 to deliver medicament 116 through needle 103 and out curved length 111 into the eyeball wall. In some embodiments, reservoir 102 has an internal volume of about 0.2 milliliters (ml). In some embodiments, reservoir 102 has an internal volume of greater than 0.2 ml. In some embodiments, reservoir 102 has an internal volume of less than 0.2 ml.

Flange 106 presents a dorsal surface 107 and a contact surface 108. Contact surface 108 is configured to physically contact the surface of the eye prior to initiating the injection. In some embodiments, contact surface 108 is curvilinear having a radius of curvature generally matching the curvature of the ocular globe of an average patient. In some embodiments, ophthalmic injector 100 is provided in several different sizes, such as for an adult human, child human, adult large animal, and so on. The radius of curvature of contact surface 108 may vary between injectors 100 in some embodiments, depending on the size of injector 100, for example. In some embodiments, contact surface 108 is a separate structure applied to flange 106 and formed from a soft, atraumatic polymer or hydrogel, so as to minimize sensation and reduce the risk of scratching or otherwise injuring the contacted surface of the eye. As can be seen in FIG. 1, for example, dorsal surface 107 buttresses and supports contact surface 108. Support of contact surface 108 is beneficial to prevent shape distortion or movement of contact surface 108 during an injection procedure.

In some embodiments, flange 106 is generally circular and has a diameter of about 75 mm. In some embodiments, the diameter of flange 106 is smaller than 75 mm. In some embodiments, the diameter of flange 106 is larger than 75 mm.

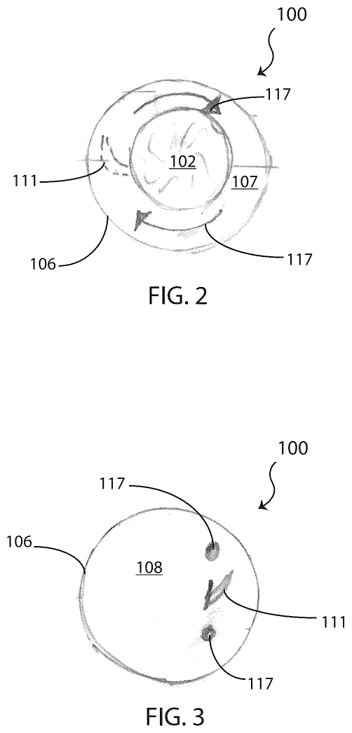

FIG. 2 is a top view of an example embodiment of an ophthalmic injection device. FIG. 2 shows ophthalmic injection device 100 with orientation markings (indicia) 117. As shown in FIG. 2, indicia 117, in some embodiments, are curvilinear arrows superimposed on dorsal surface 107 indicating the direction of rotation necessary for the user to capture the eye surface with curved length 111, such as to pierce the bulbar conjunctiva or other ocular surface. The curvilinear arrows are by example only. Other indicia may form indicia 117, in some embodiments. The position of curved length 111 beneath flange 106 is indicated by dashed lines.

FIG. 3 is a bottom view of an ophthalmic injection device. FIG. 3 shows contact surface 108 of flange 106.

Curved length 111 extends outward and externally from contact surface 108, as shown in FIG. 3. In some embodiments, curved length 111 is the curved length tip of a 30-guage needle. In some embodiments, curved length 111 is the curved length tip of a 31-guage needle. In some embodiments, curved length 111 is the curved length tip of a needle smaller than 31 gauge. In some embodiments, curved length 111 is the curved length tip of a needle larger than 30 gauge. In some embodiments, curved length 111 is embedded in the soft substance of flange 106 and requires axial pressure applied to contact surface 108, such as by the user gripping body 101 and pressing contact surface 108 onto the eyeball, to cause curved length 111 to pierce surface 108 and “capture” the cornea, or similar surface. In some embodiments, ophthalmic injector device 100 is provided with curved length 111 protruding through contact surface 108. Curved length 111 of needle 103 is configured to protrude no greater than a maximum of about 150 micrometers externally from contact surface 108, in some embodiments, whether in a fixed configuration or with application of axial pressure as described.

In some embodiments, a marking ink 115 is disposed on contact surface 108. Marking ink 115 may be disposed in a single location proximate to curved length 111, in some embodiments. In some embodiments, marking ink 115 may be disposed at two, three, or greater than three locations proximate to and surrounding curved length 111. Marking ink 115 may comprise an FDA-approved dye, such as gentian violet, for example. Other marking inks known in the art may be used. A partial listing of suitable marking inks is disclosed in U.S. Pat. No. 11,000,343, the disclosures of which are incorporated herein by reference. In some embodiments, a felt or other fabric patch saturated with marking ink 115 is coupled to contact surface 108. Injector device 100 is packaged in airtight, sterile packaging that will resist drying out of the felt patch, in some embodiments. In some embodiments, the packaging is a foil pouch. In some embodiments, a separate, removable occlusive patch is placed on contact surface 108 during manufacture and packaging of injector 100 to protect contact surface 108, marking ink 115, or both from becoming damaged or drying out during transport and storage of ophthalmic injector 100. In some embodiments, the felt patch is formed from polytetrafluoroethylene (PTFE). Other materials known in the art may be used.

In some embodiments, ophthalmic injection device 100 may be pre-packaged in a sterile, airtight foil pouch. Reservoir 102 is pre-loaded with a volume of a medicament 116, such as 0.2% lidocaine or other injectable local anesthetic, for example. To use device 100, the user removes device 100 from the sterile package and applies contact surface 108 to the conjunctival surface of a patient. The user grasps body 101 between the thumb and third finger, with the user's index finger resting lightly against reservoir 102 to stabilize device 100. The user then rotates injection device 100 while applying very gentle pressure to maintain contact between contact surface 108 and the injection surface.

In some embodiments, injection device 100 is rotated through about 20 degree of rotation. In some embodiments, injection device 100 is rotated through about 30 degrees of rotation. In some embodiments, injection device 100 is rotated through less than 20 degrees of rotation. In some embodiments, injection device 100 is rotated through greater than 20 degrees but less than 20 degrees of rotation.

In some embodiments, the user rotates body 101 fixedly coupled to needle 103 such that the user rotates the entire device 100. In some alternative embodiments, reservoir 102 is rotatably coupled to body 101 such that reservoir 102 and body 101 may be rotated independently of each other. For example, in some embodiments, the user rotates reservoir 102 is fixedly coupled to needle 103 and rotatably coupled to body 101 such that the user rotates reservoir 102 fixedly coupled to needle 103 while body 101 is not rotated. In some embodiments, user rotates body 101 fixedly coupled to needle 103 such that body 101 rotates and reservoir 102 does not rotate. In each of the aforementioned embodiments in this paragraph, reservoir 102 is rotatably coupled to body 101 allowing needle 103 to rotate either with body 101 independently from reservoir 102 or with reservoir 102 independently from body 101.

The user will detect resistance when curved length 111 has pierced and penetrated the patient's conjunctiva to the appropriate depth, which is preset according to the length and angle of curved length 111 as manufactured with device 100. The user then presses device 100 gently against the surface of the eye while compressing reservoir 102, taking care to not move contact surface 108 laterally or axially with respect to the eye surface. When reservoir 102 is fully compressed, the user then counter-rotates injector device 100 and removes device 100 from the eye injection surface.

In some embodiments wherein curved length 111 is embedded in a hydrogel, gentle downward pressure during rotation will push curved length 111 through contact surface 108 into the eye injection surface, “capturing” the injection surface. In some embodiments wherein contact surface 108, with or without marking ink 115, is covered with a removable occlusive cover, the occlusive cover is removed before placing contact surface 108 against the eye or other injection surface. In some embodiments wherein two or more marking inks 115 are disposed on contact surface 108 radially separated from curved length 111, arcuate ink marks are deposited on the conjunctive or other injection surface through contact followed by light pressure, and rotation of contact surface 108 against the injection surface. The arcuate marks accurately label the anesthetized site for the IVI, in some embodiments, making it easy for the eye surgeon to locate the anesthetized injection target site.

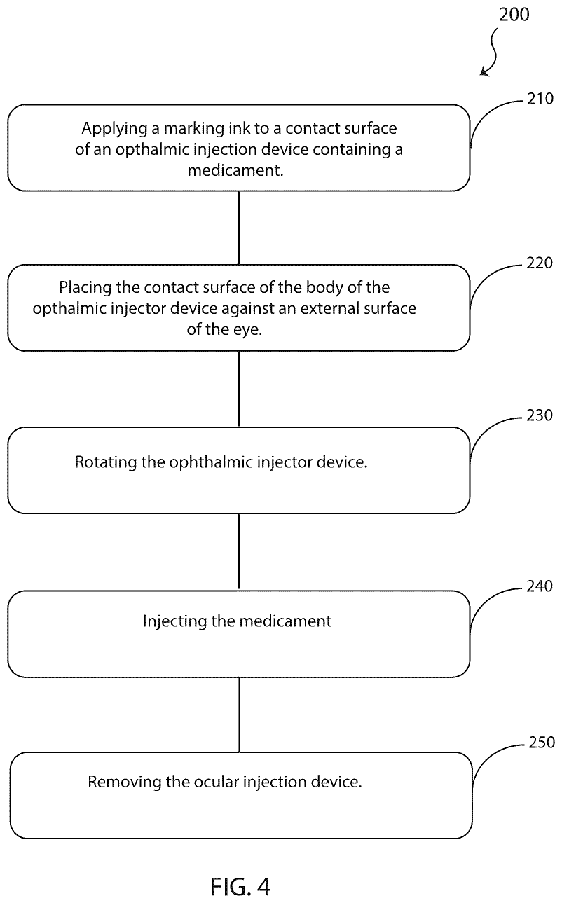

FIG. 4 is a diagram of a method of use of an ophthalmic injection device. FIG. 4 shows a method 200 comprising an applying step 210, a contacting step 220, a rotating step 230, an injecting step 240, and a removing step 250.

In some embodiments, applying step 210 comprises applying a marking ink to a contact surface of an ophthalmic injection device. Examples of types of marking inks and elements disposed on the contact surface are discussed at length herein above. Applying step 210 advantageously marks the bulbar conjunctive so that the physician performing the IVI can see exactly where a local anesthetic has been injected using device 100.

In some embodiments, contacting step 220 comprises placing a contact surface of a body of the ophthalmic injector device against an external surface of the eye, such as bulbar conjunctive covering the ocular globe. In some embodiments, the contact surface is slightly curved (concave) to generally match the external curve of the eyeball.

In some embodiments, rotating step 230 comprises rotating the ophthalmic injector device. Rotating step is performed according to the disclosures herein above, with the extent of rotation (total rotation angle) depends upon the length of the curved length of the needle and an angle formed between the curved length and the contact surface at a point where the curved length originates on the contact surface, in some embodiments.

In some embodiments, injecting step 240 comprises squeezing a pliable reservoir containing a medicament causing the medicament to pass through the needle and the curved length into the eye wall tissue.

In some embodiments, removing step 250 comprises removing the ocular injection device. To remove the device from the eye, an element of the device fixedly coupled to the curved length of needle is rotated in an angular direction opposite that of rotating step 230. When the curved length of needle has exited the eye wall, the contact surface is removed from the ocular globe.

An ophthalmic injector device and methods of use have been disclosed. The injector device facilitates participation of non-physician health care personnel in preparing the eye of a patient for an IVI procedure by simplifying establishment of local anesthesia on the conjunctive and eye wall highly desirable and generally necessary for IVI. One-handed operation allows the user to use their other hand to steady the patient's head and separate the eyelids with the user's thumb and a finger. The participation of non-physician health care staff facilitates patient flow in the office or outpatient surgical facility of a busy ocular surgeon, greatly increasing practice efficiency while simultaneously increasing safety of local anesthetic injection into the eye. The disclosed design and method of use of the ophthalmic injection device increases safety and minimizes the risk of complications, such as bleeding, conjunctival laceration, and inaccurate injection depth of conventional techniques using currently available needles, syringes, and related injection devices.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purpose of illustration and example. The description as set forth is not intended to be exhaustive or to limit the invention to the precise form disclosed. Many modifications and variations are possible, in light of the teachings set forth herein above.

What is claimed is:

1. An ophthalmic injection device comprising:
a body with an elongate shape having a flange disposed at a first end and a shoulder disposed a second end, the flange having a dorsal surface and a contact surface opposite the dorsal surface;
a pliable reservoir coupled to the shoulder; and
a needle coaxial with the body, the needle fluidly coupled to the reservoir and extending through the flange, wherein a curved length of the needle projects external to the contact surface.

2. The device of claim 1, wherein the needle is radially fixed to the body such that rotation of the body causes a corresponding rotation of the needle.

3. The device of claim 1, wherein the needle is not radially fixed to the body such that rotation of the reservoir causes rotation of the needle independent of the body and the flange contact surface.

4. The device of claim 1, wherein the flange dorsal surface bears an indicia indicating a direction of rotation to cause the needle to penetrate a tissue abutting the contact surface.

5. The device of claim 1, further comprising an orientation marking ink on the contact surface.

6. The device of claim 5, wherein the orientation marking ink is disposed at two locations on the contact surface at a location proximate to the needle curved length.

7. The device of claim 5, wherein the orientation marking ink is disposed at three locations on the contact surface proximate to the needle curved length.

8. The device of claim 1, wherein the curved length projects no greater than about 100 micrometers external to the flange contact surface.

9. The device of claim 1, wherein the body, the shoulder, and the flange are formed as a unitary structure.

10. The device of claim 1, wherein the reservoir is pre-loaded with a medicament.

11. The device of claim 10, wherein the medicament is a local anesthetic.

12. The device of claim 10, wherein a volume of the medicament is greater than or equal to about 0.1 milliliters and less than or equal to about 0.2 milliliters.

13. The device of claim 1, wherein the contact surface is curvilinear.

14. The device of claim 1, wherein the flange has a diameter of about 75 millimeters.

15. The device of claim 1, wherein the curved length of the needle is a 30 gauge needle.

16. The device of claim 1, wherein the curved length of the needle is a 31 gauge needle.

17. An ophthalmic injection device comprising:
a pliable reservoir pre-loaded with a local anesthetic medicament;
a curved length of a needle fluidly coupled to the reservoir and fixedly coupled to the reservoir; and
a body rotatably coupled to the reservoir and having a curved contact surface;
wherein fixing the contact surface and rotating the reservoir causes the curved length to enter a bulbar conjunctiva of an eye for injection of the medicament.

18. A method of using an ophthalmic injection device comprising:
applying a marking ink to a contact surface of an ophthalmic injection device containing a medicament;
placing the contact surface of the body of the ophthalmic injector device against an external surface of the eye;
rotating the ophthalmic injector device;
injecting the medicament; and
removing the ocular injection device.
